(19) United States
(12) Patent Application Publication
(10) Pub. No.: US 2004/0151778 A1
(43) Pub. Date: Aug. 5, 2004

(54) Plant protein-based microcapsules

(76) Inventors: Joel Richard, Saint-Barthelemy D'Anjou (FR); Sophie Morteau, Ifs (FR)

Correspondence Address: WINSTON & STRAWN, PATENT DEPARTMENT, 1400 L STREET, N.W., WASHINGTON, DC, 20005-3502, US

(21) Appl. No.: 10/714,347

(22) Filed: Nov. 14, 2003

Related U.S. Application Data
(63) Continuation of application No. PCT/FR02/01652, filed on May 16, 2002.

(30) Foreign Application Priority Data
May 16, 2001 (FR) 0106441

(51) Int. Cl. A61K 38/16; B01J 13/02; B01J 13/04; A61K 9/16; A61K 9/50

(52) U.S. Cl. 424/490; 264/4.1

(57) ABSTRACT

The invention relates to a method of producing microcapsules containing a material to be encapsulated. The method is characterized in that a mixture of at least one solubilized vegetable protein and a polyelectrolyte with an opposite charge to the protein is subject to complex coacervation in an aqueous medium, possibly followed by hardening, in the presence of the material to be encapsulated. The invention also relates to the microcapsules provided by the method and their uses in pharmaceutical, veterinary, cosmetic, agrofood, chemical or biomedical compositions.

### CROSS-REFERENCE TO RELATED APPLICATIONS

This application is a continuation of International application PCT/FR02/0 1652 filed May 16, 2002, the content of which is expressly incorporated herein by reference thereto.

### FIELD OF THE INVENTION

The present invention relates to a method for preparing plant protein-based microcapsules, and to the use of these capsules in the pharmaceutical, veterinary, cosmetics, agrofoods, chemical and biomedical fields.

### BACKGROUND ART

Microencapsulation includes all technologies for obtaining individualized particles, the size of which is between 1 μm and 1 mm, and which lead to the inclusion of substances or of active principles in a carrier material.

Two groups of microparticles are conventionally distinguished:

reservoir microcapsules or systems: these are spherical particles consisting of a solid envelope and a core of liquid, solid or pasty active material,

matrix microspheres or systems: they consist of a continuous network of coating material in which the substance to be encapsulated is dispersed.

Three types of encapsulation methods exist: physico-chemical methods (simple coacervation, complex coacervation, solvent evaporation, solvent extraction-evaporation, hotmelt of the coating material), chemical methods (interfacial polycondensation, polymerization in dispersed medium and gelling of the coating material) and mechanical methods (encapsulation in a fluidized air bed, by spraying and by prilling).

Complex coacervation is based on the phenomenon of desolvation of macromolecules, one positively charged and the other negatively charged, resulting in the formation of two immiscible phases, from initially homogeneous colloidal aqueous solutions. These two phases are:

the polymer-rich and water-depleted coacervate which results from the formation of complexes between the positively charged macromolecules and those which are negatively charged,

the polymer-depleted and water-rich supernatant.

The encapsulation of an oil by complex coacervation consists in emulsifying the oil in a solution of two polymers. The coacervation is induced by adjusting the pH of the medium. The polymeric complexes formed adsorb onto the droplets of oil and thus isolate them from the outside medium. The wall formed is hardened by cooling the medium and crosslinked by the action of a crosslinking agent.

The encapsulation of an active substance offers considerable advantages, such as its protection against outside agents or its slow release, delayed release or deferred release at the site of use.

For applications in the pharmaceutical, veterinary, cosmetics, agrofoods and biomedical fields, the materials most commonly desired as constituents of the wall are natural substances, in particular proteins or polysaccharides, due to their biocompatibility and their biodegradability. Among these biopolymers, albumin, gelatin, collagen and casein have been the subject of many studies.

Thus, capsules of albumin and sodium alginate have been prepared by complex coacervation in order to develop a system for encapsulating proteins and polypeptides (Singh et al., J. Pharm. Pharmacol., (1989) 41, 670-673).

Another study describes the encapsulation of an active principle in casein microspheres prepared by solvent emulsion-extraction, and it has been demonstrated that this milk protein constitutes a potential carrier for sustained-release oral preparations (Latha et al., J. Control. Rel., (1995) 34, 1-7).

For the last few years, a novel approach has consisted in using proteins of plant origin rather than animal origin. In fact, since the discovery of spongiform encephalopathy of animal origin (“mad cow” disease), consumers no longer have any confidence in products which may be contaminated by prions, the agent potentially responsible for this pathology. It is therefore becoming necessary to find a substitute for animal proteins such as gelatin and albumin. Several encapsulation techniques using these polymers of plant origin have been described in the literature.

It has been possible to encapsulate an antibiotic by simple coacervation using wheat gluten and casein as coating materials, with the aim of obtaining a sustained-release system (Jiunn-Yann Yu et al., J. of Fermentation and Bioengineering, (1997) Vol. 84, 5, 444-448)

Another controlled-release system has been developed using nanoparticles of gliadin (protein fraction of wheat gluten) obtained by a method based on desolvation of macromolecules, by addition of a protein organic phase to an aqueous phase (Ezpeleta et al., Int. J. Pharm., (1996), 131, 191-200)

Other studies have shown that it is possible to produce nanoparticles and microparticles from vicilin (pea protein) by simple coacervation (Ezpeleta et al., J. Microencapsulation, (1997), Vol. 14, No. 5, 557-565).

Finally, it is possible to prepare particles having a wall made of plant proteins using a reaction consisting of interfacial crosslinking between these proteins and a polyfunctional acylating crosslinking agent. This method makes it possible to encapsulate active substances in the solution, suspension or emulsion state, and any plant protein can be used, in particular those which are extracted from wheat, from soybean, from pea, from rapeseed, from sunflower, from barley or from oats (WO 99/03450).

As regards the complex coacervation method, the couples conventionally used are gelatin as polycation and sodium alginate, polyphosphate or gum arabic as polyanion. Studies have shown that gelatin can be substituted with bovine albumin (Singh et al., J. Pharm. Pharmacol., (1989) 41, 670-673).

Although the tendency is to use natural products of plant origin as substitutes for animal proteins, it has not been possible to carry out a method of encapsulation by complex coacervation using plant proteins. In fact, plant proteins are not pure; they present great problems of solubility due to the presence of a soluble fraction and an insoluble fraction, and also possess a low emulsifying capacity compared to that of animal proteins, which makes it necessary to use additional surfactants which interfere in the coacervate fixing phase.

Now, the inventors, surprisingly, have developed a method which solves these problems.

### SUMMARY OF THE INVENTION

The inventors have succeeded in divining a novel method which allows the use of these proteins in a complex coacervation technique to form microcapsules.

Thus, the present invention relates to a method for producing microcapsules containing a material to be encapsulated, , which comprises coacervating, in an aqueous medium and in the presence of the material to be encapsulated, a mixture of at least one solubilized plant protein and a polyelectrolyte having an opposite charge to the protein is subjected to form microcapsules comprising a complex coacervate of the plant protein and polyelectrolyte about the material to be encapsulated.

### BRIEF DESCRIPTION OF THE FIGURE

FIG. 1 shows a coacervate of SWP 100/alginate/Miglyol prior to crosslinking.

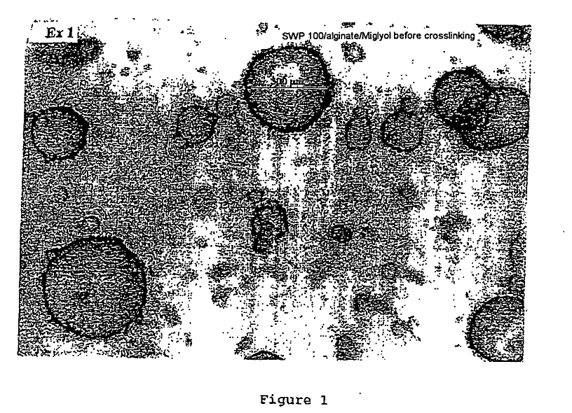

### DETAILED DESCRIPTION OF THE PREFERRED EMBODIMENTS

In a preferred embodiment of the invention, the method comprises:

a) solubilization of at least one plant protein in an aqueous medium at a pH of between 2 and 7, and below the isoelectric pH of the protein,

b) centrifugation of the solution obtained in a),

c) mixing of the supernatant obtained in b) with an aqueous solution of a polyelectrolyte with an opposite charge to that of the plant protein,

d) coacervation of the polyelectrolytes in the form of polymeric complexes, with optional hardening of the capsules, in the presence of the material to be encapsulated.

The centrifugation step b) is carried out under correctly chosen conditions, in particular at a rate of between 2,000 and 15,000 rpm, and preferably between 4,000 and 12,000 rpm, for 10 to 30 minutes.

In a particularly advantageous embodiment of the invention, the method is characterized in that the amount of soluble proteins is increased by:

e) addition of an amount of plant proteins to the supernatant with the aim of achieving saturation,

f) centrifugation of the mixture, and

g) optionally repetition of steps e) and f) several times, if desired.

Very advantageously, the method of production according to the invention is characterized in that step c) is carried out at a pH below the isoelectric pH of the plant protein, so that the protein is used as a cationic polyelectrolyte in the complex coacervation step d).

Also advantageously, the method for producing a plant protein-based microcapsule wall is characterized in that step c) is carried out at a pH above the isoelectric pH of the plant protein, so that the protein is used as an anionic polyelectrolyte in the complex coacervation step d).

The protein concentration in the initial solution is generally between 2 and 15%, the concentration of the supernatant of the initial solution of proteins centrifuged is between 1 and 8%, and the concentration of proteins in the coacervation solution is between 1.5 and 5%.

The plant proteins used in the context of the invention are extracted from plants chosen from the group comprising: lupin (genus Lupinus), soybean (genus Glycine), pea (genus Pisum), chickpea (Cicer), alfalfa (Medicago), broad bean (Vicia), lentil (Lens), bean (Phaseolus), rapeseed (Brassica), sunflower (Helianthus) and cereals such as wheat, maize, barley, malt or oats. By way of example, mention may be made of the plant proteins SWP100 and SWP50 and those marketed under the name Supro® 670 and PISANE®.

Advantageously, the anionic polyelectrolyte is chosen from those conventionally used by those skilled in the art, in particular those which are chosen from the group comprising sodium alginate, gum arabic, polyphosphates, sodium carboxymethylcellulose, carrageenan, xanthan gum and plant proteins with a pH above the isoelectric pH. Advantageously, the cationic polyelectrolyte is one of those conventionally used by those skilled in the art, in particular those which are chosen from the group comprising cationic surfactants, latexes having a quaternary ammonium, chitosan and plant proteins with a pH below the isoelectric pH.

When the method comprises a hardening step, this step can be carried out by any technique known to those skilled in the art, in particular by crosslinking with a crosslinking agent chosen from the group comprising dialdehydes such as glutaraldehyde and tannins such as tannic acid.

When the cationic polyelectrolyte is chitosan, the hardening is carried out using acetic anhydride as hardening agent.

In a particularly advantageous embodiment of the invention, use is made of a polycation/polyanion couple chosen from the group comprising the couples: SWP100/alginate, SWP100/gum arabic, chitosan/Supro®, Supro®/alginate or Supro®/gum arabic.

The microcapsules obtained by the method according to the invention have a diameter of between 5 and 500 μm, preferably 20 and 200 μm, more preferably from 20 to 50 μm.

The microcapsules according to the invention may contain substances which can be used in the pharmaceutical, veterinary, cosmetics, agrofoods, chemical and biomedical fields, and in particular active principles. They may be combined with any active ingredient or any excipient well known to those skilled in the art.

### EXAMPLES

The examples which follow illustrate the invention without, however, limiting it.

### Example 1

### Complex Coacervation Using the SWP100 Protein in the Presence of Alginate

A 10% SWP100 solution maintained at pH 3 is centrifuged for 25 minutes at 4,500 rpm. 48 ml of supernatant containing 0.72 g of dissolved proteins are obtained. 20 g of Niglyol® 812 are emulsified in this supernatant solution. 35.6 ml of an aqueous solution of sodium alginate (0.36 g) are then added followed by 96 ml of water. The temperature of the medium is 40° C. The pH of the medium is decreased from 4.22 to 3 by adding 1N hydrochloric acid.

The SWP100/alginate ratio by weight is equal to 2 and the final concentration, in the aqueous phase, of SWP100 is 0.4% weight/volume and it is 0.2% weight/volume for the alginate.

The complex coacervation takes place and the medium is cooled to 10° C. and kept at 10° C. for 1 hour. 1.5 ml of 25% glutaraldehyde are added to the medium at 10° C. The medium is then allowed to return to ambient temperature and it is kept stirring for 15 hours.

A dispersion of microcapsules containing 95% oil, with a mean size of between 200 and 400 μm, is obtained.

Microcapsules for which the SWP100/alginate ratio by weight is equal to 1 are prepared by the same technique.

### Example 2

### Complex Coacervation Using the SWP100 Protein in the Presence of Gum Arabic

A solution of 100 ml of SWP100 at 17% maintained at pH 3 is centrifuged for 25 minutes at 4,500 rpm. 100 ml of supernatant containing 2.6 g of dissolved proteins are obtained. 20 g of Miglyol® 812 are emulsified in this supernatant solution. 45 ml of an aqueous solution of gum arabic (5 g) and 13 ml of water are added. The temperature of the medium is 40° C. The pH of the medium is decreased to 3 by adding 1N hydrochloric acid.

The SWP100/gum arabic ratio by weight is equal to ½ and the final concentration of SWP100 in the aqueous phase is 1.5% weight/volume and it is 3% weight/volume for the gum arabic.

The complex coacervation takes place and the medium is cooled to 10 °C. The medium is left stirring for 1 hour and 3 ml of 25% glutaraldehyde are then added. The medium is then allowed to return to ambient temperature, still with stirring for 6 hours.

A dispersion of microcapsules containing 72.5% oil, with a mean size of between 150 and 450 μm, is obtained.

### Example 3

### Influence of Increasing the Concentration of SWP100 Protein in the Supernatant

Complex coacervation is carried out using the SWP100 protein in the presence of alginate.

The pH of a protein solution of SWP100 at approximately 15% (20 g in 130 g of water) is adjusted to a value of 4. The solution is centrifuiged a first time at 12000 rpm for 15 minutes. The pellet is removed and 12 g of SWP100 protein is added to the supernatant, the pH of which is again adjusted to 4.

A second centrifugation is performed and this operation is repeated a third time.

After the first centrifugation, the supernatant contains 2.9% of soluble protein. After three centrifugations, the soluble protein concentration is 3.6%.

The SWP100/gum arabic ratio by mass is equal to 1 and the final concentration of SWP100 and of gum arabic in the aqueous phase is 2% weight/volume.

The complex coacervation is carried out with the concentrated supernatant of SWP100 at pH 4 (100 ml containing 3.6 g of protein) and the gum arabic as anionic polyelectrolyte (80 ml containing 3.6 g of gum arabic). The coacervation is carried out according to the procedure described in Example 1.

A dispersion of microcapsules containing 73.5% oil, with a mean size of between 50 and 400 μm, is obtained.

### Example 4

### Complex Coacervation Using the Supro® 670/Alginate Couple

Capsules are prepared according to the procedure of Example 1, using a solution of Supro(® 670 made up of 22.5 g of water and 2.5 g of protein and a solution of alginate made up of 150 g of water and 1.84 g of alginate.

The coacervation pH is equal to 3.8.

Microcapsules in suspension, which contain 82% oil and which exhibit a fragile wall with a granular appearance, are obtained. The mean size of the microcapsules is between 50 and 400 μm.

### Example 5

### Evaluation of the Plant Proteins as Anionic Polyelectrolvte

A solution of Supro® 670 protein at 10% is adjusted to pH 7 and centrifuged a first time at 4,500 rpm for 25 minutes. The pellet is removed and the supernatant made up of 43 ml of water and 2.57 g of protein is used for the coacervation.

20 g of Miglyol® 812 are emulsified in the supernatant of the Supro® 670 protein solution, and then a solution of chitosan 2622 made of 120 ml of water and 1.5 g of chitosan, at pH 1.32, is added to the medium at 40° C.

The coacervation pH is adjusted to 6. The procedure is then identical to that described for Example 1.

A dispersion of microcapsules containing 83% oil, with a mean size of between 50 and 400 μm, is obtained.

What is claimed is:

1. A method for producing microcapsules containing a material to be encapsulated, which comprises coacervating, in an aqueous medium and in the presence of the material to be encapsulated, a mixture of at least one solubilized plant protein and a polyelectrolyte having an opposite charge to the protein is subjected to form microcapsules comprising a complex coacervate of the plant protein and polyelectrolyte about the material to be encapsulated.

2. The method according to claim 1, wherein the coacervating step is followed by hardening of the microcapsules.

3. The method according to claim 1, which further comprises, prior to the coacervating step:
solubilizing the at least one plant protein in an aqueous medium at a pH that is between 2 and 7 to obtain the solubilized plant protein in a solution;
centrifuging the solution to obtain a supernatant and a pellet; and
mixing the supernatant with an aqueous solution of the polyelectrolyte having the opposite charge of that of the plant protein.

4. The method according to claim 3, which further comprises increasing soluble plant proteins in the microcapsules by adding additional plant proteins to the supernatant followed by centrifuging the resultant mixture to obtain increased amounts of plant proteins in the supernatant for mixing with the polyelectrolyte, with optionally repeating of the preceding steps several times if necessary.

5. The method according to claim 3, wherein the solubilizing step is carried out at a pH below the isoelectric pH of the plant protein, so that the protein can be used as a cationic polyelectrolyte in the coacervating step.

6. The method according to claim 3, wherein the solubilizing step is carried out at a pH above the isoelectric pH of the plant protein so that the protein can be used as an anionic polyelectrolyte in the coacervating step.

7. The method according to claim 1, wherein the plant proteins are extracted from plants chosen from the group consisting of lupin (genus Lupinus), soybean (genus Glycine), pea (genus Pisum), chickpea (Cicer), alfalfa (Medicago), broad bean (Vicia), lentil (Lens), bean (Phaseolus), rapeseed (Brassica), sunflower (Helianthus) and a cereal.

8. The method according to claim 7, wherein the plant proteins are extracted from a cereal selected from the group consisting of wheat, maize, barley, malt and oats.

9. The method accordingly to claim 1, wherein the cationic polyelectrolyte is chosen from the group comprising cationic surfactants, latexes that include a quaternary ammonium, chitosan and plant proteins having a pH below the isoelectric pH.

10. The method accordingly to claim 1, wherein the anionic polyelectrolyte is chosen from the group consisting of sodium alginate, gum arabic, polyphosphates, sodium carboxymethylcellulose, carrageenan, xanthan gum and plant proteins having a pH above the isoelectric pH.

11. The method according to claim 2, wherein the hardening is carried out by crosslinking with a crosslinking agent.

12. The method according to claim 11, wherein the crosslinking agent is selected from the group consisting of dialdehydes and tannins.

13. The method according to claim 12m wherein the dialdehyde is glutaraldehyde and the tannin is tannic acid.

14. The method according to claim 2, wherein, when the cationic polyelectrolyte is chitosan, the hardening is carried out using acetic anhydride as hardening agent.

15. Microcapsules produced by the method of claim 1.

16. Microcapsules obtainable by the method of claim 1.

17. Microcapsules comprising a complex coacervate made of a mixture of plant protein and a polyelectrolyte configured encapsulating a material.

18. A pharmaceutical, veterinary, cosmetic, agrofood, chemical or biomedical composition comprising the microcapsules according to claim 15.

19. A pharmaceutical, veterinary, cosmetic, agrofood, chemical or biomedical composition comprising the microcapsules according to claim 16.

20. A pharmaceutical, veterinary, cosmetic, agrofood, chemical or biomedical composition comprising the microcapsules according to claim 17.
